(19) United States
(12) Patent Application Publication
(10) Pub. No.: US 2026/0175039 A1
(43) Pub. Date: Jun. 25, 2026

(54) LAYER RIBBON CONSTRUCTION FOR LASER RIBBON BONDING PROCESS

(71) Applicant: Cardiac Pacemakers, Inc., St. Paul, MN (US)

(72) Inventors: James Michael ENGLISH, Cahir (IE); Moira B. SWEENEY, St. Paul, MN (US); Robert O'DONOGHUE, Clonmel (IE)

(21) Appl. No.: 19/426,948

(22) Filed: Dec. 19, 2025

Related U.S. Application Data
(60) Provisional application No. 63/737,355, filed on Dec. 20, 2024.

(51) Int. Cl. A61N 1/375; A61N 1/02

(52) U.S. Cl. CPC A61N 1/37512; A61N 1/025; A61N 1/3754; A61N 1/3758

(57) ABSTRACT

An implantable medical device can include a housing including a plurality of electronic devices within the housing, wherein the electronic devices include a plurality of contacts, and a conductive ribbon attached to one of the plurality of contacts, wherein the conductive ribbon includes a bottom layer adjacent to the contact, a middle layer positioned above the bottom layer, and a top layer positioned above the middle layer; wherein the top layer is formed of a first material that couples well with an infrared (IR) laser source; wherein the middle layer is formed of a second, different material having a relatively low electrical resistance compared to the top layer and is configured to provide proper electrical performance; and wherein the bottom layer is formed of a third, different material configured to weld to the contact.

### CROSS REFERENCE RELATED APPLICATION

This application claims priority to Provisional Patent Application No. 63/737,355, filed Dec. 20, 2024, which are herein incorporated by reference in their entirety.

### TECHNICAL FIELD

Various embodiments described herein relate to apparatus, systems, and methods associated with implantable medical devices.

### 1BACKGROUND

An ambulatory medical device, such as an implantable medical device (IMD), can be configured for implant in a subject, such as a patient. An IMD can be configured to be coupled to a patient's heart such as via one or more implantable leads. Such an IMD can obtain diagnostic information or generate therapy to be provided to the patient, such as via the coupled implantable lead.

IMDs can include a header that is coupled to a housing that houses much of the electronics of the IMD. The header is electrically connected to electronics in the housing by a feedthrough assembly. The housing can further include a plurality of electronic devices and components attached to a PCB, for example. There is a need to ensure robust electrical connections between the various electrical contacts of the IMD.

### SUMMARY

Example 1 can include subject matter such as an implantable device. The implantable medical device can include a housing including a plurality of electronic devices within the housing; wherein the electronic devices include a plurality of contacts; and a conductive ribbon attached to one of the plurality of contacts, wherein the conductive ribbon includes a bottom layer adjacent to the contact, a middle layer positioned above the bottom layer, and a top layer positioned above the middle layer; wherein the top layer is formed of a first material that couples well with an infrared (IR) laser source; wherein the middle layer is formed of a second, different material having a relatively low electrical resistance compared to the top layer and is configured to provide proper electrical performance; and wherein the bottom layer is formed of a third, different material configured to weld to the contact.

In Example 2, the subject matter of Example 1 can optionally include wherein the top layer is a material with relatively high absorption of IR energy from the IR laser source.

In Example 3, the subject matter of any one or more of Examples 1-2 can optionally include wherein the top layer includes Ni or Nb.

In Example 4, the subject matter of any one or more of Examples 1-3 can optionally include wherein the material of the top layer further encases both side surfaces of the ribbon.

In Example 5, the subject matter of any one or more of Examples 1-4 can optionally include wherein middle layer includes a metal or alloy including copper or silver.

In Example 6, the subject matter of any one or more of Examples 1-5 can optionally wherein the bottom layer includes a metal or alloy that is miscible with the contact when the laser source is applied.

In Example 7, the subject matter of any one or more of Examples 1-6 can optionally include wherein the bottom layer includes a metal or alloy including gold or platinum.

In Example 8, the subject matter of any one or more of Examples 1-7 can optionally include wherein the bottom layer includes a material that is immiscible with the contact.

In Example 9, the subject matter of any one or more of Examples 1-8 can optionally include wherein the top layer has a thickness between 10-40% of an overall thickness of the ribbon, the middle layer has a thickness of between 45-75% of the overall thickness, and the bottom has a thickness of between 5-25% of the overall thickness.

In Example 10, the subject matter of any one or more of Examples 1-9 can optionally include wherein the top layer has a higher melting point than the bottom and middle layers.

In Example 11, the subject matter of any one or more of Examples 1-10 can optionally include wherein the housing is electrically coupled to a header by a feedthrough wire running from a feedthrough assembly to a PCB, and wherein the contacts are positioned upon the PCB within the housing.

Example 12 can include a conductive ribbon for providing an electrical connection within an implantable medical device. The conductive ribbon can include a bottom layer adjacent to a contact, a middle layer positioned above the bottom layer, and a top layer positioned above the middle layer; wherein the top layer is formed of a first material that couples well with an infrared (IR) laser source; wherein the middle layer is formed of a second, different material having a relatively low electrical resistance compared to the top layer and is configured to provide proper electrical performance; and wherein the bottom layer is formed of a third, different material configured to weld to the contact.

In Example 13, the subject matter of any one or more of Examples 1-12 can optionally include wherein the top layer is a material with relatively high absorption of IR energy from the IR laser source.

In Example 14, the subject matter of any one or more of Examples 1-13 can optionally include wherein the top layer includes Ni or Nb.

In Example 15, the subject matter of any one or more of Examples 1-14 can optionally include wherein the material of the top layer further encases both side surfaces of the ribbon.

In Example 16, the subject matter of any one or more of Examples 1-15 can optionally include wherein the middle layer includes a metal or alloy including copper or silver.

In Example 17, the subject matter of any one or more of Examples 1-16 can optionally include wherein the bottom layer includes a metal or alloy that is miscible with the contact when the laser source is applied.

In Example 18, the subject matter of any one or more of Examples 1-17 can optionally include wherein the bottom layer includes a metal or alloy including gold or platinum.

Example 19 can include subject matter such as a method of attaching a connector ribbon to contact of an implantable medical device. The method can include placing a conductive ribbon upon a contact, wherein the conductive ribbon includes a bottom layer adjacent to a contact, a middle layer positioned above the bottom layer, and a top layer positioned above the middle layer, wherein the top layer is formed of a first material that couples well with an infrared (IR) laser source, wherein the middle layer is formed of a second, different material having a relatively low electrical resistance compared to the top layer and is configured to provide proper electrical performance, and wherein the bottom layer is formed of a third, different material configured to weld to the contact; and applying an IR laser weld to the ribbon.

In Example 20, the subject matter of any one or more of Examples 1-19 can optionally include wherein the top layer is a material with relatively high absorption of IR energy from the IR laser source.

In Example 21, subject matter (e.g., a system or apparatus) may optionally combine any portion or combination of any portion of any one or more of Examples 1-20 to comprise “means for” performing any portion of any one or more of the functions or methods of Examples 1-20, or at least one “non-transitory machine-readable medium” including instructions that, when performed by a machine, cause the machine to perform any portion of any one or more of the functions or methods of Examples 1-20.

This summary is intended to provide an overview of subject matter of the present patent application. It is not intended to provide an exclusive or exhaustive explanation of the disclosure. The detailed description is included to provide further information about the present patent application. Other aspects of the disclosure will be apparent to persons skilled in the art upon reading and understanding the following detailed description and viewing the drawings that form a part thereof, each of which are not to be taken in a limiting sense.

### 1BRIEF DESCRIPTION OF THE DRAWINGS

FIG. 1 shows an example implantable medical device, in accordance with one embodiment.

FIG. 2 shows a side view of a PCB, in accordance with one embodiment.

FIG. 3 shows a cross-section view of a connector ribbon, in accordance with one embodiment.

FIG. 4 shows a cross-section view of a connector ribbon, in accordance with one embodiment.

FIG. 5 shows a schematic representation of a welded connector ribbon, in accordance with one embodiment.

FIG. 6 shows a method of attaching a connector ribbon to contact of an implantable medical device, in accordance with one embodiment.

### 1DETAILED DESCRIPTION

1In the following detailed description, reference is made to the accompanying drawings that form a part hereof and in which are shown, by way of illustration, specific embodiments in which the invention may be practiced. These embodiments are described in sufficient detail to enable those skilled in the art to practice the invention. Other embodiments may be utilized and structural, logical, and electrical changes may be made.

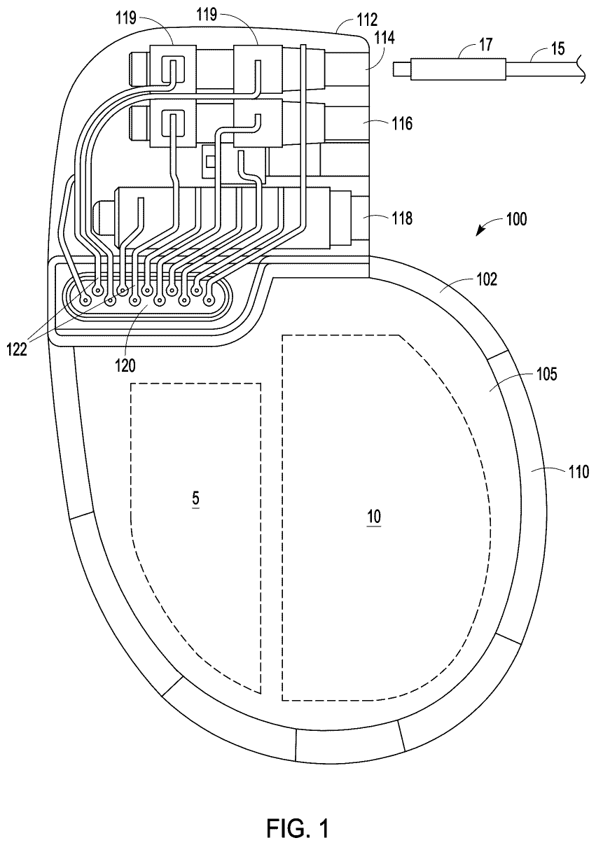

FIG. 1 shows an implantable system 100 including an implantable medical device 102, in accordance with one embodiment. The implantable medical device 102 includes a pulse generator 105 and at least one implantable lead 15. The pulse generator 105 includes a housing 110 and a header 112 mounted to the housing 110. The pulse generator 105 can be implanted into a subcutaneous pocket made in the wall of a patient's chest. Alternatively, the pulse generator 105 can be placed in a subcutaneous pocket made in the abdomen, or in other locations. The pulse generator 105 can include electronic devices such as a power supply 5 including a battery, a capacitor, and other components housed in the housing 110. The pulse generator 105 can further include other electronic devices including electronic components 10, such as microprocessors to provide processing, evaluation, and to deliver electrical shocks and pulses of different energy levels and timing for defibrillation, cardioversion, and pacing to a heart in response to cardiac arrhythmia including fibrillation, tachycardia, heart failure, and bradycardia.

The header 112 can include one or more bores 114, 116, 118 to receive the implantable lead 15. The implantable lead 15 can include electrodes on a distal end to provide therapy to a body and include a terminal pin 17 on a proximal end to couple to one of the bores 114, 116, 118. At least one electrical conductor is disposed within the lead 15 and extends from the proximal end to the electrode. The electrical conductor carries electrical currents and signals between the pulse generator 105 and the electrode.

Contacts on the terminal pin 17 can electrically contact electrical one or more contacts 119 within the bores 114, 116, 118 to allow signals and therapy to be delivered to and from electrodes in the body to the electronics 5, 10 within the housing 110. The contacts 119 can be connected by a plurality of wires 122 to a feedthrough assembly 120 to electrically communicate between the lead 15 and the electronics within the housing 110.

As noted, the pulse generator 105 includes electronic devices including the power supply 5 and the electronic components 10, such as microprocessors, to provide processing, evaluation, and to deliver electrical shocks and pulses of different energy levels and timing for various conditions.

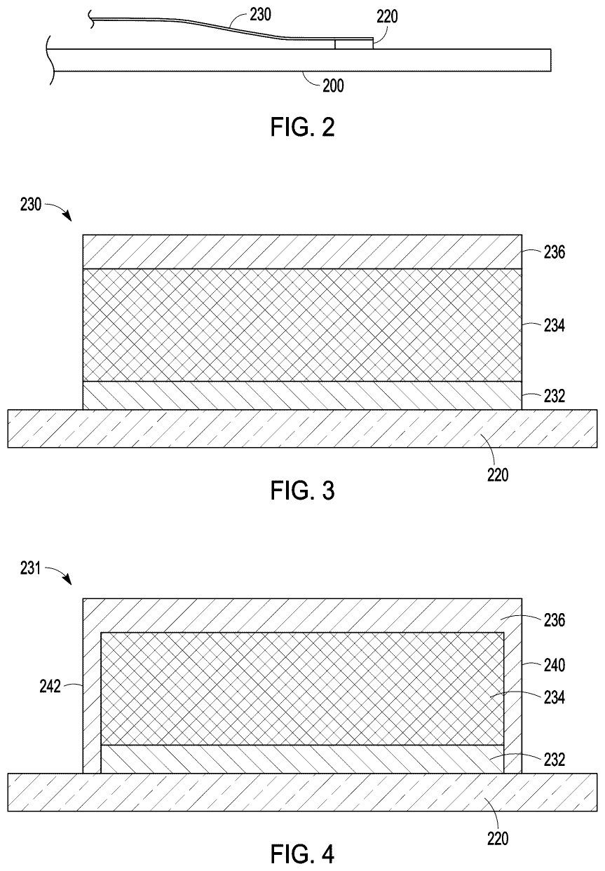

FIG. 2 shows a top view of a portion of a PCB 200, in accordance with one embodiment.

Here, a portion of a printed circuit board assembly (PCB 200) within the housing 110 is represented. The PCB 200 can include a plurality of electrical contacts 220. Further the feedthrough assembly 120 and the batteries and capacitors (FIG. 1) of the IMD can include such contacts. Connector ribbons 230 can be used to couple between various of the contacts, such as contact 220, and other contacts of the device. The PCB 200 can include a plurality of electronic components 10 connected to the PCB 200 and can include a plurality of contacts 220. For the sake of clarity, only one contact and one connector ribbon 230 is shown, In actual use, the PCB 200 can include a plurality of all the items.

As noted above, there is a need to ensure robust connections between the various electrical contacts of the IMD. One method to bond a connector ribbon 230 to the contact 220 can include laser welding. However, laser ribbon bond welding of micro interconnects with in IMD can be a challenge.

For example, many of the contacts, such as contact 220, can be connected to heat sensitive components, thus the energy applied by the laser should be as low as possible. However, the ribbon connector 230 material needs to be of low resistance. But such materials are not compatible with generally available laser sources.

Other problems can include that a number of different contact substrate materials that need to be welded. Moreover, space is of a premium for both the ribbon dimensions, along with the substrate weld surface. Also, fatigue and bending stiffness compete against the laser welding of the joint due to requiring too much heat energy. To meet all these competing challenges, design trade-offs need to be made.

The present system helps overcome one or more of these challenges by using a connector ribbon construction which can be formed using three or more metals (or alloys) in a layered construction.

FIG. 3 shows a cross-section view of a multi-layer connector ribbon 230, in accordance with one embodiment.

In this example, the conductive ribbon 230 includes a multi-layer construction including a bottom layer 232 adjacent to the contact 220, a middle layer 234 positioned above the bottom layer 232, and a top layer 236 positioned above the middle layer 234.

In this example, the top layer 236 can be formed of a first material that couples well with an infrared (IR) laser source, for example. In other words, the top layer is chosen from the materials that have a high absorption rate with an IR laser source. The middle layer 234 can be formed of a second, different material having a relatively low electrical resistance compared to the top layer 236 and is configured to provide proper electrical performance for the IMD, and the bottom layer 232 can be formed of a third, different material configured to properly weld to the substrate contact 220.

Thus, the multi-layer construction provides that the top layer 236 material is chosen to enable a stable interaction with the IR laser and the middle and bottom layers 234, 323 are chosen to enable high conductivity. Accordingly, the multi-layer ribbon 230 enables IR welding of a low resistivity interconnect.

Moreover, as will be discussed, another factor is that the selected materials of the different layers can have different melting points with the top layer material having a higher melting point temperature than the lower layers. This also can improve the quality of the bond since the laser weld energy will pass through and melt the lower layers without melting the top layer.

As noted, it is desired that the material of the top layer 236 enable stable interaction with the laser source. Specifically, the top layer 236 should have low reflectivity of the laser light (i.e., a relatively high absorption of IR energy from an IR laser source), and low resistivity. Thus, the top layer 236 can be chosen from the group of materials with good conductivity and good absorption of IR wavelengths. In one example, the top layer 236 can be selected to be a material which couples with the laser source well, such as Ni or Nb (however, it is not limited to these two materials). For example, Nb has a higher melting point relative to lower layers 232, 234, and good absorption of the IR wavelengths. Moreover, Nb can be a useful material for the top layer 236 since it resists weld splatter and has a high surface tension.

In other embodiments, the top layer 236 can be include one or more materials and alloys chosen from the group including niobium (Nb), titanium (Ti), nickel (Ni), Monel, MP35N, and stainless steel.

It should be noted that, in some examples, a green or blue laser can be utilized for forming the ribbon connection. In such a case, a material for the top layer 236 can be chosen that absorbs the green or blue light more readily, such as copper or silver or the like, for example.

In this embodiment, the middle layer 234 can be a material which is of low resistance which is good for the electrical performance of the system, i.e. copper (Cu) or silver (Ag), or the like.

In other embodiments, the middle layer 234 can be include one or more materials and alloys chosen from the group including copper, gold (Au), aluminum (Al), nickel, silver, platinum (Pt), palladium (Pd), and Monel.

The bottom layer 232 can include a metal or alloy that is preferably miscible with the contact 220 when the laser weld is applied. The typical contact 220 is a gold-coated copper contact. Thus, a the bottom layer 232 material is selected from the group of materials that forms a robust weld (preferably miscible) with such a contact. However, in some examples, depending on the material of the contact 220, the weld may be immiscible.

Thus, the bottom layer 232 can be selected to be a metal or alloy which will ready weld to the contact substrate material, and in some cases, it may form eutectic alloys. For example, the bottom layer 232 can include a material such as gold or platinum, or the like. In one example, the bottom layer 232 can include a eutectic alloy of 65% Cu/35% Au.

In other embodiments, the bottom layer 232 can be include one or more materials and alloys chosen from the group including copper, gold, aluminum, nickel, silver, platinum, palladium, and Monel.

As noted, the surface contact 220 can include a copper substrate with a gold-coated contact surface. However, other contacts within the IMD can include a metal or alloy including one or more of nickel, niobium, titanium, stainless steel, gold, copper, aluminum, silver, platinum, palladium, Monel, MP35N, tungsten, indium, tin lead alloy (solder), and iron.

The material of the bottom layer 232 can be chosen to have good wettability with the chosen substrate material such that, when welded, the bottom layer 232 spreads evenly over the substrate surface.

The specific materials given above are only some examples of usable layer materials. In various embodiments, the various materials for the top layer 236, bottom layer 232, and middle layer 234 can be chosen using phase diagrams and material laser weld compatibility charts.

Overall, the ribbon 230 can have a thickness of about 0.025 inches (0.635 mm). The top layer 236 can have a thickness of between 10-40% of the overall thickness of the ribbon 230, the middle layer 234 can have a thickness of between 45-75% of the overall thickness, and the bottom layer 232 can have a thickness of between 5-25% of the overall thickness. In one example the top, middle, and bottom layers can take up 25%, 62.5%, and 12.5% of the overall thickness, respectively.

The technology to create such a layered ribbon 230 is known in the art. For example, cladding is one example of a suitable technology to make such a ribbon.

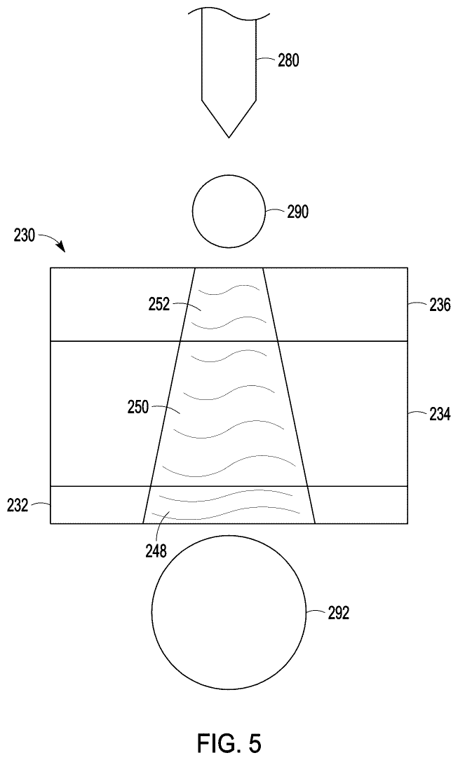

FIG. 4 shows a cross-section view of a connector ribbon 231, in accordance with one embodiment. The ribbon 231 of FIG. 4 is similar to the ribbon 230 discussed in FIG. 3. Thus, the above discussion applies to this example, and only the substantive differences will be discussed.

In this embodiment, the top layer 236 can be extended down the sides 240, 242 of the ribbon 230 to help control the weld pool growth, especially when eutectic alloys are been formed in the weld joint area between the ribbon 230 and the contact 220. Thus, the material of the top layer 236 can encase the side surfaces of the ribbon 230, without covering the bottom of the ribbon 230.

FIG. 5 shows a schematic representation of a welded connector ribbon 230, in accordance with one embodiment.

As schematically shown in FIG. 5, an IR laser from a laser source 280 propagates through the combination of materials of the ribbon 230 such that a cross section area of a weld joint 292 at a bottom of the ribbon is greater than a cross-section of a weld pool 290 at the top of the ribbon 230. In contrast, in a typical laser ribbon weld the weld energy dissipates, forming an inverse reflow pyramid such that the top weld pool cross-section area is much greater in size than the weld joint cross section area at the bottom. By using the dissimilar materials of the present ribbon 230 as discussed above, the top layer 236 absorbs the IR energy without a high degree of melting, while the other layers have lower melting points. Accordingly, the result is that the overall weld reflow of the ribbon 230, including a top weld reflow 252, a middle weld reflow 250, and a bottom weld reflow 248 define an overall generally pyramidic shape such that the cross section area of the weld joint 292 at the bottom of the ribbon is greater than a cross-section of the weld pool 290 at the top of the ribbon 230. This provides for a more robust welded connection to the contact substrate compared to a typical laser ribbon weld using the same amount of energy.

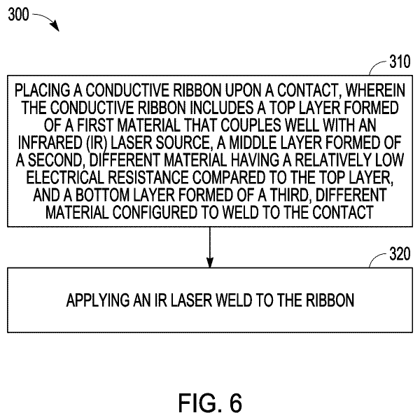

FIG. 6 shows a method (300) of attaching a connector ribbon to contact of an implantable medical device, in accordance with one embodiment.

Referring to the ribbons 230, 231 discussed above, the method (300) can include placing a conductive ribbon upon a contact (310). The conductive ribbon can include a bottom layer adjacent to a contact, a middle layer positioned above the bottom layer, and a top layer positioned above the middle layer. The top layer can be formed of a first material that couples well with an infrared (IR) laser source, the middle layer can be formed of a second, different material having a relatively low electrical resistance compared to the top layer and is configured to provide proper electrical performance, and the bottom layer can be formed of a third, different material configured to weld to the contact.

The method can then include applying an IR laser weld to the ribbon (320).

In various examples, the method can further include where the top layer is a material with relatively high absorption of IR energy from the IR laser source.

For the reasons discussed above, in one embodiment the top layer can include Ni or Nb, the middle layer can include copper or silver, and the bottom layer can include gold. In one example, the bottom layer 232 can include a eutectic alloy of 65% Cu/35% Au.

As discussed, multiple different materials (listed above) can be used in each layer. In order to select the correct combination of materials, phase diagrams and material laser weld compatibility matrix charts can be used.

In summary, the present system helps overcome one or more of the challenges of laser ribbon bonding by using a ribbon construction which can be formed using three or more metals (or alloys) in a layered construction.

A top layer of the ribbon can be formed of a first material that couples well with an infrared (IR) laser source. The middle layer 234 can be formed of a second, different material having a relatively low electrical resistance compared to the top layer and is configured to provide proper electrical performance for the IMD, and the bottom layer 232 can be formed of a third, different material configured to properly weld to the substrate contact 220.

Thus, the multi-layer construction provides that the top layer 236 material is chosen to enable a stable interaction with the IR laser and the middle and bottom layers 234, 323 are chosen to enable high conductivity. Accordingly, the multi-layer ribbon 230 enables IR welding of a low resistivity interconnect.

### ADDITIONAL NOTES

1The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim. 1Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

What is claimed is:

1. An implantable medical device comprising:
a housing including a plurality of electronic devices within the housing;
wherein the electronic devices include a plurality of contacts; and
a conductive ribbon attached to one of the plurality of contacts, wherein the conductive ribbon includes a bottom layer adjacent to the contact, a middle layer positioned above the bottom layer, and a top layer positioned above the middle layer;
wherein the top layer is formed of a first material that couples well with an infrared (IR) laser source;
wherein the middle layer is formed of a second, different material having a relatively low electrical resistance compared to the top layer and is configured to provide proper electrical performance; and
wherein the bottom layer is formed of a third, different material configured to weld to the contact.

2. The implantable medical device of claim 1, wherein the top layer is a material with relatively high absorption of IR energy from the IR laser source.

3. The implantable medical device of claim 2, wherein the top layer includes Ni or Nb.

4. The implantable medical device of claim 1, wherein the material of the top layer further encases both side surfaces of the ribbon.

5. The implantable medical device of claim 1, wherein the middle layer includes a metal or alloy including copper or silver.

6. The implantable medical device of claim 1, wherein the bottom layer includes a metal or alloy that is miscible with the contact when the laser source is applied.

7. The implantable medical device of claim 6, wherein the bottom layer includes a metal or alloy including gold or platinum.

8. The implantable medical device of claim 1, wherein the bottom layer includes a material that is immiscible with the contact.

9. The implantable medical device of claim 1, wherein the top layer has a thickness between 10-40% of an overall thickness of the ribbon, the middle layer has a thickness of between 50-75% of the overall thickness, and the bottom has a thickness of between 5-25% of the overall thickness.

10. The implantable medical device of claim 1, wherein there is a relatively high melt differential between the top layer and the bottom and middle layers.

11. The implantable medical device of claim 1, wherein the housing is electrically coupled to a header by a feedthrough wire running from a feedthrough assembly to a PCB, and wherein the contacts are positioned upon the PCB within the housing.

12. A conductive ribbon for providing an electrical connection within an implantable medical device, the conductive ribbon comprising:
a bottom layer adjacent to a contact, a middle layer positioned above the bottom layer, and a top layer positioned above the middle layer;
wherein the top layer is formed of a first material that couples well with an infrared (IR) laser source;
wherein the middle layer is formed of a second, different material having a relatively low electrical resistance compared to the top layer and is configured to provide proper electrical performance; and
wherein the bottom layer is formed of a third, different material configured to weld to the contact.

13. The conductive ribbon of claim 12, wherein the top layer is a material with relatively high absorption of IR energy from the IR laser source.

14. The conductive ribbon of claim 13, wherein the top layer includes Ni or Nb.

15. The conductive ribbon of claim 12, wherein the material of the top layer further encases both side surfaces of the ribbon.

16. The conductive ribbon of claim 12, wherein the middle layer includes a metal or alloy including copper or silver.

17. The conductive ribbon of claim 12, wherein the bottom layer includes a metal or alloy that is miscible with the contact when the laser source is applied.

18. The conductive ribbon of claim 17, wherein the bottom layer includes a metal or alloy including gold or platinum.

19. A method of attaching a connector ribbon to contact of an implantable medical device, the method comprising:
placing a conductive ribbon upon a contact, wherein the conductive ribbon includes a bottom layer adjacent to a contact, a middle layer positioned above the bottom layer, and a top layer positioned above the middle layer, wherein the top layer is formed of a first material that couples well with an infrared (IR) laser source, wherein the middle layer is formed of a second, different material having a relatively low electrical resistance compared to the top layer and is configured to provide proper electrical performance, and wherein the bottom layer is formed of a third, different material configured to weld to the contact; and
applying an IR laser weld to the ribbon.

20. The method of claim 19, wherein the top layer is a material with relatively high absorption of IR energy from the IR laser source.
